(19) United States
(12) Patent Application Publication
(10) Pub. No.: US 2025/0347669 A1
(43) Pub. Date: Nov. 13, 2025

(54) SYSTEM AND METHOD OF CALIBRATING SENSING INSTRUMENTS

(71) Applicant: THE ARMSTRONG MONITORING CORPORATION, Ottawa (CA)

(72) Inventors: Joanne JOHNSON, Ottawa (CA); Stephanie PICHETTE, Ottawa (CA); Rodney SPRULES, Ottawa (CA); Curtis TOUPIN, Ottawa (CA); Aaron WATKINS, Ottawa (CA); Jean BISSON, Ottawa (CA)

(21) Appl. No.: 18/871,088

(22) PCT Filed: May 17, 2023
(86) PCT No.: PCT/CA2023/050675

§ 371 Date: Dec. 2, 2024

Related U.S. Application Data
(60) Provisional application No. 63/348,793, filed on Jun. 3, 2022.

(51) Int. Cl. G01N 33/00

(52) U.S. Cl. CPC G01N 33/0006

(57) ABSTRACT

A calibration system and method for calibrating an instrument are provided. The system comprises at least one sensor, a processor, and a memory comprising instructions which, when executed by the processor, configure the processor to perform the method. The method comprises obtaining a series of sensor readings, determining variations between changes in successive (or near successive) sensor readings from the series of sensor readings, estimating a stabilization point of the sensor readings by identifying at least one gas sensor reading from series of sensor readings at which increases in sensor readings and decreases in sensor readings are approximately offsetting such that the slope of the trend line is near zero, and adjusting a parameter in the instrument that represents an association between sensor readings and a known physical quantity based on the stabilization point.

### CROSS-REFERENCE TO RELATED APPLICATIONS

This claims the benefit of U.S. Provisional Patent Application No. 63/348,793, filed Jun. 3, 2022, the entire contents of which are incorporated by reference herein.

### FIELD

The present disclosure relates generally to systems and methods of calibrating sensing instruments.

### BACKGROUND

Gas sensors degrade in terms of signal output and drift through time and therefore should be calibrated regularly. Several challenges are often encountered in doing this calibration. First, the sensor response time and signal output vary depending on the environment within which they operate; i.e., they are affected by temperature, humidity, radio frequency interference (RFI), the presence of other gases, etc. Second, given that sensor signals approach the maximum output asymptotically, different service technicians might wait different (and insufficient or excessive) amounts of time to enable the sensor to reach the output level required for calibration. Third, the output and the speed to maximum output vary from sensor to sensor, even amongst the same model by the same manufacturer.

There are several methods that can be used in sensor calibration. One common calibration method involves waiting until the maximum output (or minimum output in the case of a sensor with reverse polarity) is achieved and calibrating the sensor (i.e., adjusting the gain) accordingly. There are many associated problems with this waiting period, given that the signal approaches a maximum asymptotically. Firstly, the technician might not wait long enough. Additionally, the longer it takes to wait for maximum output, the more toxic or combustible gas that is consumed, which is expensive in terms of both labour and gas consumption and which results in prolonged release of toxic or combustible gases into the air, with a potential negative impact on the environment. Another potential calibration method is determining the T80 or T90, defined as the time at which the sensor's response to the gas is 80% or 90% of the final response, respectively, which involves the exposure of the sensor to gas until it reaches 80% or 90% of its maximum output, and extrapolates the necessary gain. Consequently, while this method is much faster than waiting until the maximum output is achieved, the results are much less accurate and may lead to over-reporting and costly false alarms. Moreover, because the sensor output degrades through time, and T80 and T90 also vary through time, the sensitivity of the sensor is unknown with this procedure.

Calibration routines have also been developed based on analysis of when the change in readings becomes small, relative to some pre-defined threshold. However, because both the speed and magnitude of sensor response is impacted by a variety of factors, a different threshold would be optimal for every environment (i.e., every possible permutation of temperature, humidity, pressure, presence of other gases, etc.), which is not practical. Consequently, the use of the change relative to some pre-defined threshold would be lower than optimal for some situations and higher than optimal for other situations.

Because sensor output is impacted by temperature, humidity and other factors, the maximum (or minimum) expected output, as well as the length of time necessary to reach those, also varies by those factors. Lookup tables are frequently created for temperature or humidity, based on specifications provided by sensor manufacturers, but manufacturers generally do not provide output data by varying combinations of temperature and humidity (and do not provide look-up tables for all the potential influencing factors). It is possible to conduct research and gather data for numerous combinations of temperature and humidity points, but this is very time consuming and still subject to human error.

### SUMMARY

In some embodiments, there is provided a calibration system for calibrating an electronic instrument. The calibration system comprises at least a sensor, a processor and a memory comprising instructions which when executed by the processor configure the processor to obtain a series of sensor readings, determine variations between successive (or near successive) sensor readings, estimate a stabilization point of the sensor readings by identifying a “stability” region where increases in sensor readings and decreases in sensor readings are approximately offsetting such that the slope of the trend line is near zero. More precisely, the region of stability occurs when the sum of the increases between successive or near successive readings is approximately equal to the absolute value of the sum of the decreases between successive readings; equally, the sum of increases between successive readings is about 50% of the sum of the absolute value of the differences between successive readings. In this embodiment a stabilization of the gas sensor readings point is estimated by identifying at least one gas sensor reading from series of gas sensor readings at which the sum of increases between successive readings equates to about 50% of the sum of the absolute value of the differences between successive readings, and a parameter in the instrument that represents an association between sensor readings and a known physical quantity of gas based on the stabilization point is adjusted accordingly.

In some embodiments, there is provided another calibration system for calibrating an electronic instrument. The calibration system comprises at least one gas sensor, a processor, and a memory comprising instructions which when executed by the processor configure the processor to obtain a series of gas sensor readings, determine variations between changes in successive or near successive gas sensor readings from the series of gas sensor readings, estimate a stabilization point of the gas sensor readings by identifying at least one gas sensor reading from series of gas sensor readings at which the sum of increases between successive readings equates to about 50% of the sum of the absolute value of the differences between successive readings, and adjust a parameter in the instrument that represents an association between sensor readings and a known physical quantity of gas based on the stabilization point. There can be a tolerance for this proportion, (e.g., +/−0.025). This tolerance can be a function of various parameters such as the local mean and standard deviation of the variations, and/or any tolerance range suitable based on an analysis of the data (e.g., measured empirically), selected based on the target being measured (e.g., gas, humidity, pressure, etc.), selected base on a type of the target being measured (e.g., based on the severity of the type of gas, the setting, etc.).

In some embodiments the calibration system comprises a processor, and a memory comprising instructions as well as a temperature sensor, and/or a humidity sensor, and/or a pressure sensor and/or a vibration sensor and/or a motion sensor and/or a light and/or a sound sensor and/or a particulate matter sensor. In these embodiments, as described in the ones above, the calibration system obtains a series of sensor readings, determine variations between changes in successive or near successive sensor readings from the series of sensor readings, estimates a stabilization point of the sensor readings by identifying at least one sensor reading from a series of sensor readings at which the sum of increases between successive readings is about 50% of the sum of the absolute value of the differences between successive readings from at least one previous sensor reading is within a range of values or sufficiently close to a value (e.g., 0.5) representative of a stable signal, and adjusts a parameter in the instrument that represents an association between sensor readings and a known physical quantity based on the stabilization point.

In some embodiments, there is provided a method for calibrating an instrument. The method comprises obtaining a series of sensor readings, determining variations between changes in successive or near successive sensor readings from the series of sensor readings, estimating a stabilization point of the sensor readings by identifying at least one sensor reading from series of sensor readings the sum of increases between successive readings equates to about 50% of the sum of the absolute value of the differences between successive readings, and adjusting a parameter in the instrument that represents an association between sensor readings and a known physical quantity based on the stabilization point.

In various further aspects, the disclosure provides corresponding systems and devices, and logic structures such as machine-executable coded instruction sets for implementing such systems, devices, and methods.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the instant disclosure.

### DESCRIPTION OF THE FIGURES

Embodiments will be described, by way of example only, with reference to the attached figures, wherein in the figures:

FIG. 1 illustrates, in a schematic, an example of a calibration system for calibrating an instrument, in accordance with some embodiments;

FIG. 2 illustrates, in a flowchart, an example of a method of calibrating an instrument, in accordance with some embodiments;

FIG. 3 illustrates, in a flowchart, an example of a method of calibrating a gas sensor, in accordance with some embodiments;

FIG. 4 illustrates, in a flowchart, an example of a method of gathering the buffering data and receiving input, in accordance with some embodiments;

FIG. 5 illustrates, in a flowchart, an example of a method of finding an initial, “pre-gassing”, stable reading, in accordance with some embodiments;

FIG. 6 illustrates, in a flowchart, an example of a method of waiting for gas (or other target phenomena), in accordance with some embodiments;

FIG. 7 illustrates, in a flowchart, an example of a method of finding a final, “post-gassing”, stable reading, in accordance with some embodiments;

FIG. 8 illustrates, in a flowchart, an example of a method of determining grade of observation, in accordance with some embodiments;

FIG. 9 illustrates, in graphs, examples of sensor response versus time, in accordance with some embodiments;

FIG. 10 illustrates, in a schematic, a detection system which reacts to a signal or stimulus from a physical phenomenon, in accordance with some embodiments;

FIG. 11 is a schematic diagram of a computing device such as a server; and

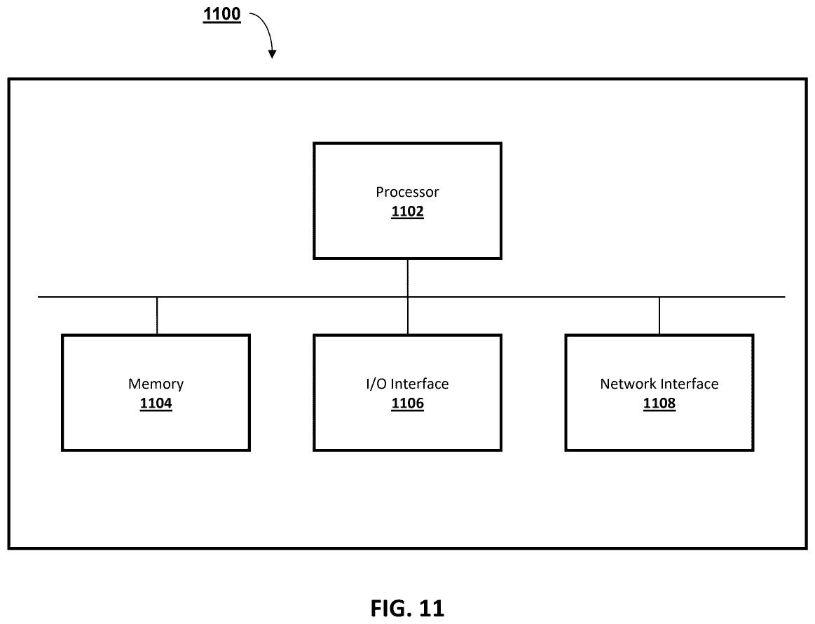

FIG. 12 shows a schematic representation of the system chip, as a combination of software and hardware components in a computing device.

It is understood that throughout the description and figures, like features are identified by like reference numerals.

### DETAILED DESCRIPTION OF THE EMBODIMENTS

One objective of the present disclosure is to develop a method of calibrating sensing instrumentation (such as a gas sensor, a temperature sensor, a humidity sensor, a pressure sensor, a vibration sensor, a motion sensor, a light sensor, a sound sensor, a particle sensor, a biosensor and/or any sensor in an electronic detection or sensing instrument) that is:
Fast
Precise and consistent
Automatically adjusts to drive gassing, or application of other target phenomena for the appropriate amount of time
Gathers and leverages data on factors that might impact gas (or other phenomena) readings, such as temperature, humidity, vibration, pressure, etc.
Requires little computational resources, data storage and/or power

It is also worth noting that the smaller the data storage and power requirement, the more feasible it is do the calibration on the site where a phenomena (e.g., gas) is being detected.

Typically, a sensor signal is approximately normally distributed around the trend line. Consequently, when the standard deviation of the signal is less than or approximately equal to the standard deviation of the signal at an earlier time, the signal can be said to be stabilizing. In previous teachings, a method of calibrating sensing instrumentation (such as a gas sensor, a temperature sensor, a humidity sensor, a pressure sensor, a vibration sensor, a motion sensor, a light sensor, a sound sensor, a particulate matter sensor, a biosensor and/or any sensor in an electronic detection or sensing instrument) was provided, referred to as RADiCal. This method uses an estimate of the standard deviation of variations inherent in the sensor signal in order to self-assess when the sensor output reaches a level close enough to its maximum (or minimum in the case of decreasing signal) and calculates the adjustment required for calibration. More specifically, standard deviations can be used as a measure of the small, random variations inherent in the signal, and stability is measured where the calculated slope of the signal (or slope changes) is smaller than some number of standard deviations. While this method is effective, this method may require modification and updates in response to hardware and firmware changes such as sampling frequency than the method provided herein and tends to be more computationally intensive than the method proposed herein.

In some embodiments, a computationally inexpensive indicator of stability is provided, without suffering from many of the challenges associates with the impacts on sensor readings noted above (such as variances due to temperature, humidity, pressure, variance between sensor elements, age of the sensor, etc.).

Specifically, for a given series of successive or near successive signal output readings S₁, S₂, . . . , Sₙ the following indicator is used:

$$Approximate Movement Indicator ( AMI ) = \frac{\sum {U}_{i}}{\sum {U}_{i} + \sum {D}_{i}}$$

where

$$\{ {U}_{i} \} = \{ {S}_{i} - {S}_{i - 1} ⋁ {S}_{i} > {S}_{i - 1} \}$$

$$and$$

$$\{ {D}_{i} \} = \{ {S}_{i - 1} - {S}_{i} ⋁ {S}_{i} < {S}_{i - 1} \} .$$

The AMI is an index is different from the relative strength indicator (RSI) that measures the magnitude of price changes by using the ratio of the average of price increases relative to the average of price decreases over a specified number of days in the following way:

$$RSI = 100 - ( \frac{100}{1 + RS} )$$

$$where RS = ( \frac{Averageofxdays ’ upcloses}{Averageofxdays ’ downcloses} ) .$$

It should be noted that there are notable important differences between AMI and RSI. For example, the behaviour of the data and intended objective for the use of the RSI for the stock market is different than is the AMI for use in the calibration of instruments.

The stock market rises and falls in waves—there is no predetermined expect pattern to the curve (other than an expected positive change over the very long run and an oscillation around the trendline, but even the slope of the trend line changes over time). Rising (“Bull”) markets tend to last longer than slow or declining (“Bear”) markets, but with no pre-determined time for each. Furthermore, the stock market does typically move with momentum, so while its “hot”, it keeps increasing up until some event or series of events causes a downturn, in which case it will keep declining until another event happens. In addition, as technology changes and certain industries evolve and grow, while others mature or decline, the underlying assets also grow at different rates. In summary, the RSI is negatively serially correlated around the trend line over the very long term, but positively serially correlated around the trend line over the short term, and the variations are neither random, nor normally distributed.

In the calibration of instruments, the expected general shape of the curve is known, and the oscillations around that shape are generally random and close to normally distributed with mean of 0 and a roughly constant standard deviation. Because of the changes typically being random and normally distributed around the trendline, the following can be inferred:
whenever the AMI is 1 the sensor is on the upward portion of the curve (for an increasing sensor);
when the AMI is 0, its on the decreasing portion of its curve (for a reducing sensor); and
when the AMI is close to 0.5, it is in a stable region where the increases and decreases are attributable to the random, normally distributed fluctuations around the trend line.

In some embodiments, variations (e.g., small “random” variations) inherent in the signal are used as a way to self-assess when the sensor has reached an output level that is close enough to its maximum (or minimum in the case of a sensor which produces a decreasing signal) that it can be used to calculate the adjustment required for calibration. For example, an initial reference point (for example the signal at a zero gas concentration) and the “near” maximum at a non-zero gas concentration point are estimated by finding the output where the AMI is sufficiently close to a value (e.g. 0.5) representative of a stable signal over a given set of samples. As another example, the initial reference point and the “near” maximum might pertain to temperature in degrees centigrade, relative humidity measurements, vibration (rate of change in displacement per unit time), pressure (Pascal), frequency of intensity of light or sound waves, the number of particles, etc.

It should be understood that the terms ‘near maximum’ or ‘near zero’ include readings that are approximately near the actual maximum reading or zero reading, respectively. In some embodiments, the near maximum or near zero may include the actual maximum or actual zero reading, respectively. It should also be understood that the ‘near maximum’ or ‘near minimum’ may pertain to the first or second difference of the change (i.e., where the slope begins to increase, stabilize, or hit an inflection point or some other pattern or range of response). It should also be understood that references to approximate limits to gas (or other phenomena) readings (i.e., near maximum, near minimum, near zero) in this disclosure may be different for different gases (or other phenomena being measured). It should also be understood that throughout this disclosure, references to the terms ‘maximum’, ‘minimum’ and ‘zero’ include ‘near maximum’, ‘near minimum’ and ‘near zero’.

In some embodiments, the outputs at any two known reference values are used by finding the output where percentage of recent signal change which was in the upward direction is sufficiently close to a value representative of a stable signal. In some embodiments, calibrations may be performed with a range different than the minimum and the maximum. For example, this approach would apply with an oxygen sensor whose first reference is the background concentration and secondary reference is 0% by volume.

In some embodiments, target gas already exists in the ambient are, and thus is already influencing the gas sensor reading. In this embodiment, the person calibrating the equipment would use another device (typically a portable) to estimate the background gas, enter the value of the background gas in upon initiating the calibration routine, find stability within this environment of background gas, then apply gas and find stability at the (typically higher) known concentration of gas.

In another embodiment, the point (and associated output) at which “near stability” has been achieved can be approximated by identifying the point at which the percentage of recent signal change which was in the upward direction is sufficiently close to a value representative of a stable signal. In one embodiment, this point is found where the ratio of the absolute sum of positive first differences between two output readings a pre-defined distance apart to the absolute sum of all first differences between two output readings the same pre-defined distance apart a pre-defined number of observations into the past is sufficiently close to a value representative of a stable signal for a predefined minimum number of periods. This is then repeated ensuring that all such readings are separated by at least a pre-defined number of samples. This is just one example of how stability can be measured using the variations inherent in the signal, and there are other algorithms or approaches that could be taken.

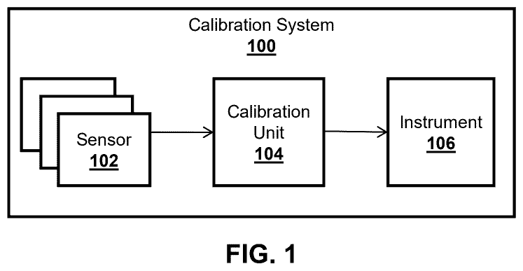

FIG. 1 illustrates, in a schematic, an example of a calibration system 100 for calibrating an instrument, in accordance with some embodiments. The system 100 comprises at least one sensor 102, a calibration unit 104, and an instrument 106. In some embodiments, the at least one sensor 102 may be one or more gas sensors, temperature sensors, humidity sensors, pressure sensors, vibration sensors, motion sensors, light sensors, sound sensors, and/or particle sensors. Other components may be added to the system 100, including one or more amplifiers. As will be described in more detail below, the calibration unit 104 receives sensor readings from the sensor 102 and adjusts or calibrates a parameter in the instrument 106. For example, the received sensor readings may be gas sensor readings in the case where the at least one sensor 102 is a gas sensor. In some embodiments, the sensor 102 and/or the calibration unit 104 may be a component of instrument 106. Other components may be added to the system 100, including one or more amplifiers.

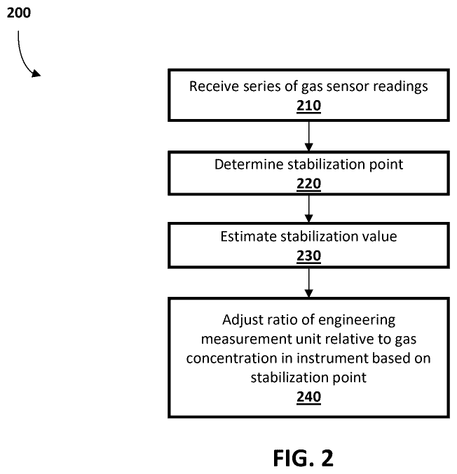

FIG. 2 illustrates, in a flowchart, an example of a method 200 of calibrating an instrument, in accordance with some embodiments. The method 200 may be performed by the calibration unit 104 or an instrument 106 that includes logic performed by the calibration unit 104. The method 200 includes obtaining 210 a series of sensor readings. i.e., the calibration unit 104 logic receives readings from the sensor 102 and/or instructs a device or instrument having a sensor 102 to obtain the reading. Variations between changes in successive sensor readings from the series of sensor readings may then be determined 220. A characterization point, such as a stabilization point, of the sensor readings may then be estimated 230 by identifying at least one sensor reading from the series of sensor readings at which the total positive change in successive or near successive sensor readings over some period of time is about equal to the total negative change in successive or near successive sensor readings over that same period. Once the stabilization point is estimated 230, a parameter representing an association between sensor readings and a known physical quantity (in some embodiments, a target value may represent the physical quantity) in the instrument is adjusted 240. In some embodiments, this comprises adjusting, in the instrument 106, a ratio of an engineering measurement unit relative to the known physical quantity. In some embodiments this ratio may be adjusted in the firmware. In other embodiments, this ratio may be adjusted by changing the physical gain in one or more amplifiers. In still other embodiments, this ratio may be adjusted through a combination of firmware and physical gain adjustments of one or more amplifiers. Other steps may be added to the method 200.

In some embodiments, the sensor readings in FIG. 2 may be sensors 102 and sensor readings pertain to temperature sensors, humidity sensors, pressure sensors, vibration sensors, motion sensors, light sensors, sound sensors, and/or particle sensors. The physical quantity in the association being adjusted is relative to the type of sensor 102. For example, for a gas sensor, the physical quantity is a gas concentration level.

In some embodiments, the system 100 may comprise, and method 200 may apply to, more than one different type of sensor 102. In such embodiments, each type of sensor 102 may obtain separate measurements and stored in different memory files. The method 200 steps may be applied separately to those separate measurements independent from the other measurements. The system 100 may be configured to calibrate one parameter pertaining to one type of sensor at a time, or different parameters for different sensors in parallel (but separate) applications of the method 200.

The remaining methods will be described for gas sensors for ease of presentation. However, it should be understood that the following methods may also apply to different types of sensors with appropriate modifications. For example, the phenomenon being measured and the physical quantity being assessed can be replaced with that which applies to the different type of sensor. i.e., references to gas sensors or measurements or other readings pertaining to gas sensors may be replaced, mutatis mutandis, with those that apply to a different type of sensor (whether or not this is explicitly indicated below).

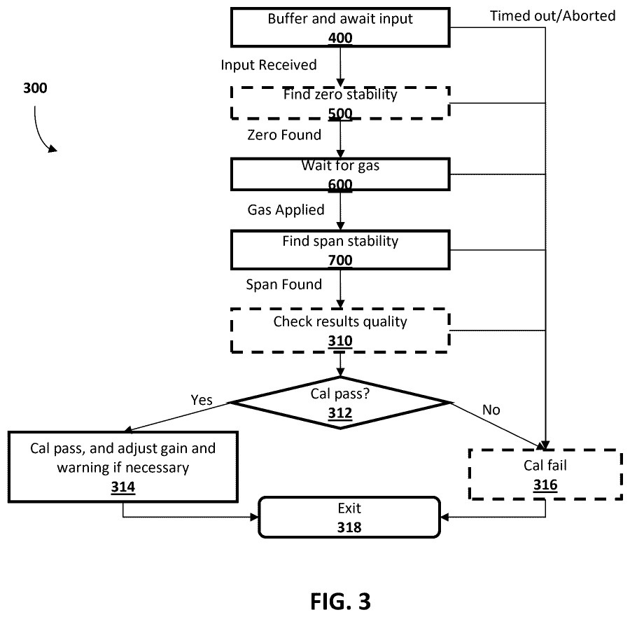

FIG. 3 illustrates, in a flowchart, another example of a method 300 of calibrating an instrument, in accordance with some embodiments. The method 300 may be performed by the calibration unit 104 or an instrument 106 that includes logic performed by the calibration unit 104. FIG. 3 shows the high-level steps (described in greater detail below) involved in calibrating a gas sensing instrument 106 using variations inherent in the signal. The method 300 comprises gathering sample data in a buffer and receiving input 400, (optionally) determining a stable zero output level 500, waiting for gas (or other external stimulus) from a physical system to be applied 600, and determining the stable signal span 700. Optionally, the quality of the sensor output may be checked 310. Once the span is determined 700 (or the sensor output is checked 310), if the calibration passed 312, then the instrument is adjusted 314. Otherwise 312, the calibration failed and the calibration mode is exited 316. It should be understood that a calibration can pass or fail. In order to “pass” a calibration attempt results are to meet a predetermined expectation. If not, then the calibration attempt would be considered as “fail” which means the results of the calibration would not be saved.

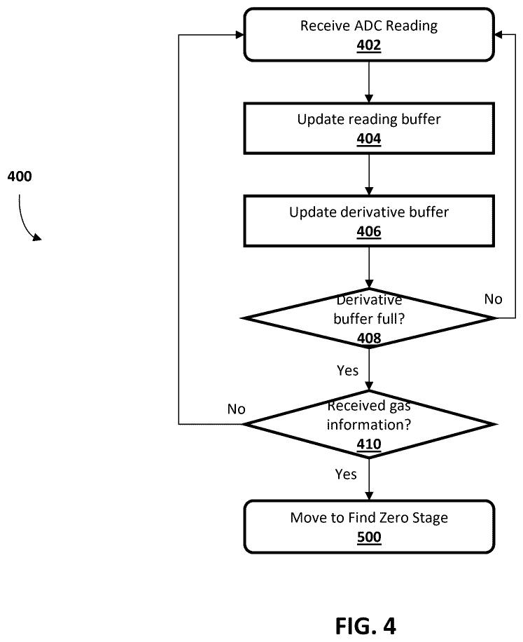

FIG. 4 illustrates, in a flowchart, an example of a method 400 of gathering the buffering data and receiving input, in accordance with some embodiments. The method 400 may be performed by the calibration unit 104 or an instrument 106 that includes logic performed by the calibration unit 104. The gas sensing instrument 106 is put into calibration mode, and begins receiving 402 the signal from the sensor 102 representing an analog-to-digital converter (ADC) reading. In some embodiments, the calibration mode involves having logic similar to that performed by the calibration unit 104 in the instrument 106. In other embodiments, the calibration mode may involve placing a device including the calibration unit 104 receiving readings from a gas sensor 102 associated with an instrument 106. The instrument 106 continues receiving data 402 which is passed to the calibration unit 104. The calibration unit 104 updates a reading buffer 404, which then also propagates to a buffer of the first differences between successive or near successive readings in the reading 406. In some embodiments, first differences of filtered near successive readings or first differences of successive or near successive average readings will be saved in the buffer. Once the derivative buffer 408 is full, the calibration unit 104 checks to see if it has received the required gas (or other target phenomena) information 410, which may include but is not limited to the gas concentration (or other physical quantity), for calibration, background gas (or other physical quantity), temperature, humidity and/or other factors which are known to affect (i.e., amplify, reduce or otherwise excite the signal). It should be noted that in some embodiments, the reading buffer and the derivative buffer may comprise one or more of the same or different buffers. If the reading buffer is full 408, and the calibration unit 104 has received the required gas information 410, it 104 will move on to the next stage (which in this example is the optional Find Zero Stability stage 500 of FIG. 3 (or any initial starting point), but which could be the Waiting for Gas (or some other target phenomena or reading) 600 stage of FIG. 3 if some pre-determined zero were to be used). If the calibration were to be done with other hardware 108, the additional hardware 107 would need to receive gas readings for a predefined period, or until the additional hardware 108 tells the additional hardware 107 to stop receiving gas.

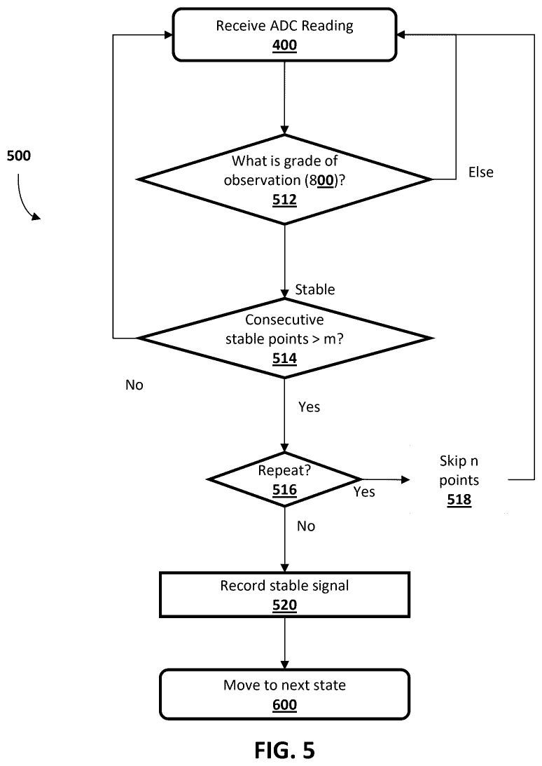

FIG. 5 illustrates, in a flowchart, an example of a method 500 of finding an initial, “pre-gassing” stable reading, in accordance with some embodiments. Note that the parameters such as thresholds discussed herein may differ from the later stage of determining a final or “post-gassing” stable reading, for example. These parameters can be chosen based on predictions from a theoretical model or by experimentation. These parameters influence the likelihood of finding stabilization when the signal is still moving and the expected time required for a stabilization to be found. Changing parameters to decrease the likelihood of finding stabilization when the signal is still moving will typically have effect of increasing the expected time required for stabilization. The method 500 may be performed by the calibration unit 104 or an instrument 106 that includes logic performed by the calibration unit 104. The method 500 comprises the sensing instrument 106 (gas or other) which is put into calibration mode, and begins receiving 402 the signal from the sensor 102. The calibration unit 104 continues receiving data 402 and proceeds to determine the grade of the observation 512 with a first known concentration of gas (or any other target phenomena) applied. This first concentration may be the concentration of gas (or other target phenomena) which is present in the environment at the time of calibration. If unstable, the calibration unit 104 will return to receiving the data 402 and continue until a stable point is read 512. Optionally, (to increase the requirement for stability), if stable, the calibration unit 104 may proceed to determine if the number of consecutive stable observations is greater than a predefined threshold 514; otherwise, step 514 may be skipped and proceed directly to 516. If the threshold is passed, a determination is made as to whether this process should be repeated 516. If it is to be repeated a pre-determined number following observations are disregarded, and the process repeats again 518. If it is not to be repeated the stable signal is recorded 520 and the calibration unit 104 moves to the next state 600, otherwise the calibration unit 104 returns to the beginning of the process and receives the next observation 402.

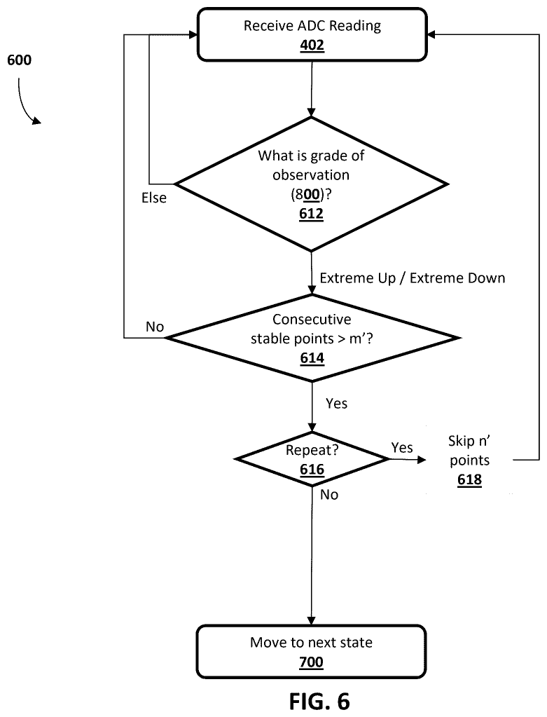

FIG. 6 illustrates, in a flowchart, an example of a method of detecting when gas (or other target phenomena) has been applied to the sensor 600, in accordance with some embodiments. The method 700 may be performed by the calibration unit 104 or an instrument 106 that includes logic performed by the calibration unit 104. The method 600 comprises the sensing instrument 106 (gas or other) which is put into calibration mode, and begins receiving 402 the signal from the sensor 102. The instrument 100 continues receiving data 402 and proceeds to determine the grade of the observation 612. Optionally, if the data passes an extreme change in value, the calibration unit 104 could proceed to determine if the number of consecutive similar observations is greater than a predefined threshold 614; otherwise it could proceed to step 616, or step 616 could be skipped as well and that unit could proceed directly to 700. If the threshold is passed, a determination is made as to whether this process should be repeated 616. If it is to be repeated a pre-determined number following observations are disregarded, and the process repeats again 618. If it is not to be repeated the calibration unit will recognize that gas is being applied and will move on to the next state 700, otherwise the calibration unit 104 returns to the beginning of the process and receives the next observation 402.

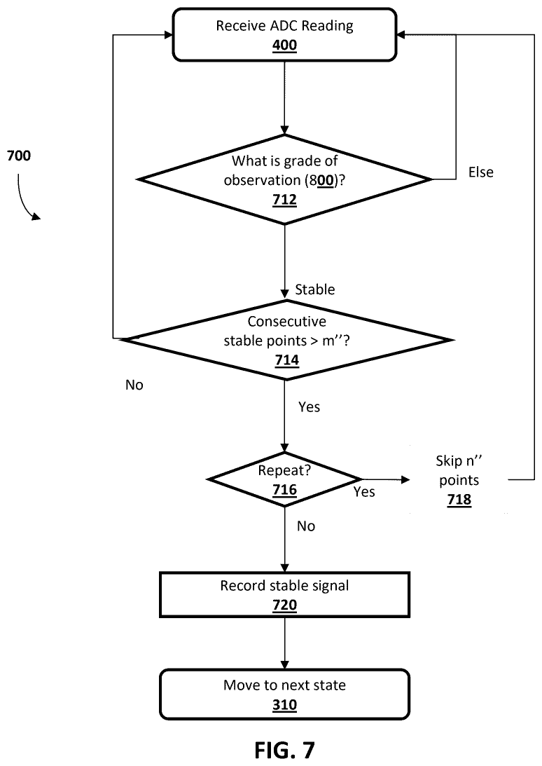

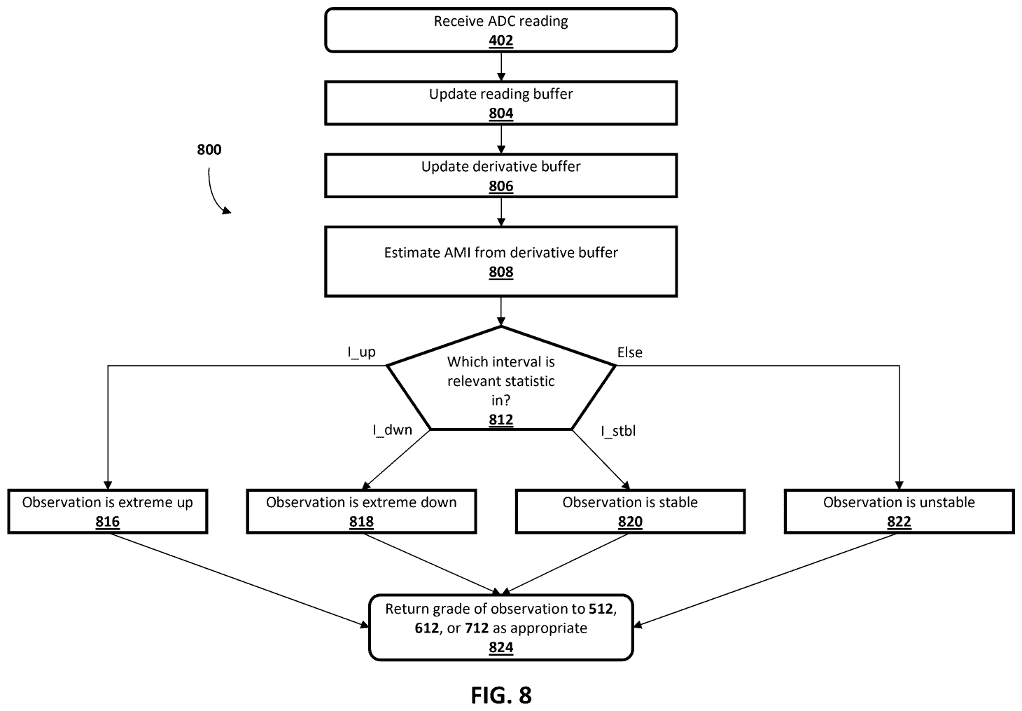

FIG. 7 illustrates, in a flowchart, an example of a method 700 of finding a final, “post-gassing” stable reading, in accordance with some embodiments. It should be noted that the parameters such as thresholds discussed herein may differ from the prior stage wherein an initial, “pre-gassing” stable reading is found, for example. These parameters can be chosen based on predictions from a theoretical model or by experimentation. These parameters influence the likelihood of finding stabilization when the signal is still moving and the expected time required for a stabilization to be found. Changing parameters to decrease the likelihood of finding stabilization when the signal is still moving will typically have effect of increasing the expected time required for stabilization. The method 700 may be performed by the calibration unit 104 or an instrument 106 that includes logic performed by the calibration unit 104. The method 500 comprises the sensing instrument 106 (gas or other) which is put into calibration mode, and begins receiving 402 the signal from the sensor 102. The calibration unit 104 continues receiving data 402 and proceeds to determine the grade of the observation 712 with a second known concentration of gas (or any other target phenomena) applied. If unstable, the calibration unit 104 will return to receiving the data 402 and continue until a stable point is read 712. Optionally, if stable, the calibration unit 104 could proceed to determine if the number of consecutive stable observations is greater than a predefined threshold 714 for added certainty of stability. If the threshold is passed, a determination is made as to whether this process should be repeated 716. If it is to be repeated a pre-determined number following observations are disregarded, and the process repeats again 718. If it is not to be repeated the stable signal is recorded 720 and the calibration unit 104 moves to the next state 310, otherwise the calibration unit 104 returns to the beginning of the process and receives the next observation 402. FIG. 8 illustrates, in a flowchart, an example of a method of determining the grade of an observation 800, in accordance with some embodiments. The method 800 may be performed by the calibration unit 104 or an instrument 106 that includes logic performed by the calibration unit 104. The method 800 illustrates one embodiment of the detailed steps in determining the grade of the observation for the purpose of estimating the span and gain adjustment. The grade of the observation is determined using a pre-determined statistic calculated from the small random variations inherent in the signal. In some embodiments, this statistic is the percentage of total recent movement which was in an upward direction In some embodiments, the standard deviation may be used as an additional criteria for determining an acceptable level of stability in addition to, or in place of a pre-defined tolerance range. For example, as the sensor ages, the variations may not fall within two standard deviations 95% of the time without any serial correlation, and as such, the standard deviation may be used to fail calibration because of the standard deviation estimate. Alternatively, the standard deviation may be retained from the initial calibration and compared to a future calibration where if the standard deviation of the signal has varied by more than a predetermined amount, then the signal is not stable enough. In any case, once a statistic is chosen the stochastic properties of this statistic can be determined and analyzed using techniques familiar to those knowledgeable in the field.

This embodiment involves receiving the ADC reading 402 and storing it into the reading buffer 804. The reading is then used to populate the derivative buffer 806. Next the AMI is estimated using the values recorded from the derivative buffer. It should be noted that if all values in the derivative buffer are zero, the AMI is taken to be 0.5 808. In one embodiment, sensor readings are classified into one of four grades based on the value of the AMI. For brevity, we will call these grades extreme up, extreme down, stable and unstable. The extreme grades would be expected when there is a rapid change in signal, such as immediately after gas (or other physical quantity) is applied to the sensor. The stable grade would be expected when the system has been allowed enough time to reach equilibrium. The unstable grade would be expected during the interim, where the change in the signal is neither rapid enough to be considered extreme nor slow enough to be considered stable.

In some embodiments, we define an observation to be stable if its AMI is at most a pre-determined threshold, a, away from 0.5 820. Similarly, we define an observation to be extreme if its AMI is at most another pre-determined threshold, away from either 1 or 0 depending on the direction of the signal 816, 818. If neither of these conditions are met, the observation is considered unstable 822. The parameters, a and B can be chosen based on predictions from a theoretical model or by experimentation. Decreasing B will decrease the sensitivity to change in the signal before determining that a change in concentration has been observed. Decreasing a will increase the specificity of the algorithm in determining when the signal has stabilized and consequently increase the expected time required for a stabilization region to be found. It should be noted that choices of a and B may vary between different stages of the calibration process. For example, a may be different while determining an initial, “pre-gassing” stable reading 500 than it is when determining a final, “post-gassing” stable reading 700. Finally, the grade of the sample is communicated to the instrument 824.

For greater clarity, a common use case for gas sensing is described as follows. A parking garage will frequently have a CO detecting instrument in it. Common alarm levels might be 25 PPM (which would activate the HVAC system to dissipate or expel the gas) and 100 PPM (which would generate an audible and visual alarm to advise occupants. Upon initial factory calibration, a reading of 100 PPM might be associated with an ADC count of 2800. The sensor signal output often declines at a rate of 2% per month. Hence if an instrument was calibrated in January, by June the instrument might only be reading 88 PPM when shown 100 PPM of gas. Consequently, a field service technician would indicate to the transmitter that they were going to begin calibration, expose the sensor to 100 PPM of gas, and wait until the instrument advised that a stabilization point had been found. For example, if a is 0.025 and B is 0.05, in the early part of gassing, the slope of the signal would be steep and therefore the signal's AMI would be between 0.95 and 1 (or between 0 and 0.05 for a reducing sensor), respectively. Therefore, the reading would be determined to be extreme. Eventually, as gassing continued and the slope tapers off, the AMI would be between 0.475 and 0.525, the signal would be graded as stable, and the corresponding ADC count would be recorded. If the corresponding ADC count was 2400, the firmware would then update the system's memory to reflect that 100 PPM of gas was associated with the 2600 ADC counts. When the sensor next encountered gas that caused it to reach the new ADC count recorded in memory, it would activate the HVAC system.

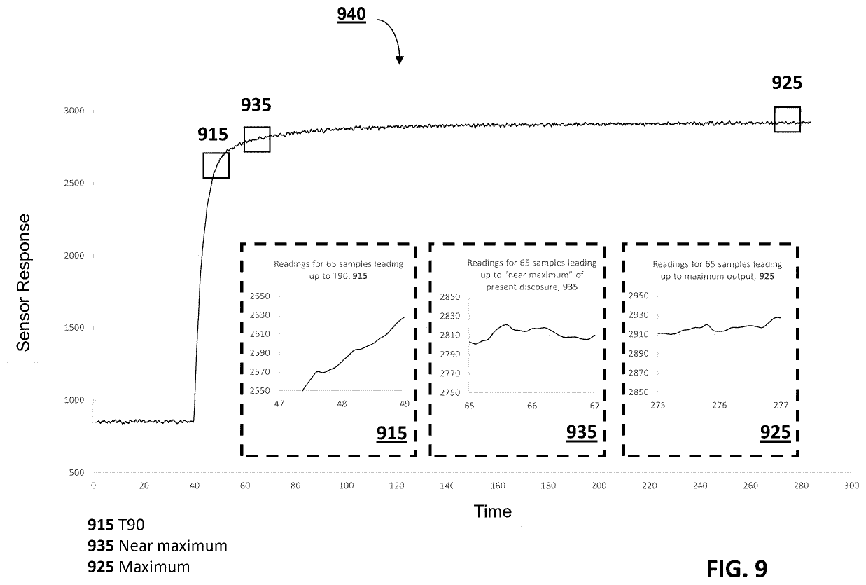

FIG. 9 illustrates, in a graph 940, examples of sensor response versus time, in accordance with some embodiments. Prior methods using T90 (915) and Maximum Output (925) are compared with the example of sensor response versus time for the method described herein (935). The total upward variation between the beginning and end of over a similar time range for T90 915, is not matched by the total downward variation. In the case of the present disclosure near maximum 935, the response is still increasing, as it asymptotically approaches its absolute maximum, but the change is sufficiently small so as to be considered an approximation of a near maximum. Therefore, the present disclosure offers a method for identifying the first point in which the variation over a range is less than the variation within a range of successive, or near-successive samples by self-referencing the historical successive variations.

The example above illustrates the concept of using self-referencing historical variations to calibrate detection instrumentation. One skilled in the art would appreciate the similarities with the more complex methodology used in the calibration of detection instrumentation described herein.

The embodiments of the disclosure described above offer a number of benefits, which can be illustrated through analysis of a series of examples. The examples below pertain to CO, NO₂ and Oxygen, but relate more generally to any type of gas sensor. The above method is readily extended to new types of sensors (including non-gas sensors), so long as one is familiar with the variations inherent in the signal patterns inherent to that type of sensor.

### Speed.

As shown in a series of examples presented in Table 1 below, this calibration method returns output that is 95.0% to 99.2% of the near maximum output in typically less than one quarter (e.g. 34.3 to 50.1 seconds for CO for the AMI version, compared to between 194.1 and 220.9 seconds for full max). Table 1 shows an example of average statistics by calibration method.

TABLE 1
Average statistics by calibration method
Average Values

| | Carbon Monoxide | | | | | | Nitrogen Dioxide | | Oxygen | | | |
|---|---|---|---|---|---|---|---|---|---|---|---|---|
| Serial Number | 012A | 012B | 013A | 013B | 014A | 014B | 004A | 005A | 015A | 016A | 017A | 018A |
| Count of gas type | 132 | 110 | 141 | 110 | 53 | 52 | 95 | 107 | 61 | 63 | 117 | 125 |
| Output at zero gas | | | | | | | | | | | | |
| sensor.response.min | 853 | 883 | 861 | 883 | 710 | 712 | 2,053 | 1,932 | 1,770 | 1,794 | 1,814 | 1,851 |
| radical.min | 857 | 886 | 864 | 891 | 711 | 714 | 2,053 | 1,931 | 1,770 | 1,794 | 1,815 | 1,850 |
| ami.min | 857 | 886 | 864 | 891 | 712 | 715 | 2,053 | 1,932 | 1,770 | 1,794 | 1,815 | 1,850 |
| Sensor response at | | | | | | | | | | | | |
| sensor.response.max | 2,924 | 2,956 | 2,849 | 2,829 | 2,810 | 2,813 | 1,444 | 1,326 | 2,705 | 2,730 | 2,607 | 2,567 |
| sensor.response.at.t80 | 2,510 | 2,541 | 2,451 | 2,440 | 2,390 | 2,393 | 1,566 | 1,447 | 2,518 | 2,543 | 2,449 | 2,424 |
| sensor.response.at.t90 | 2,717 | 2,749 | 2,650 | 2,634 | 2,600 | 2,603 | 1,505 | 1,386 | 2,612 | 2,637 | 2,528 | 2,496 |
| radical.max | 2,894 | 2,930 | 2,822 | 2,804 | 2,786 | 2,780 | 1,452 | 1,331 | 2,697 | 2,723 | 2,605 | 2,564 |
| ami.max | 2,823 | 2,861 | 2,761 | 2,748 | 2,731 | 2,712 | 1,461 | 1,336 | 2,693 | 2,719 | 2,600 | 2,561 |
| radical.percent.of.max | 98.4 | 98.6 | 98.5 | 98.4 | 98.8 | 98.3 | 98.5 | 99.1 | 99.1 | 99.2 | 99.6 | 99.7 |
| ami.percent.of.max | 95.0 | 95.2 | 95.5 | 95.6 | 96.1 | 95.1 | 97.0 | 98.2 | 98.7 | 98.7 | 99.1 | 99.2 |
| Span | | | | | | | | | | | | |
| sensor.response.change | 2,071 | 2,073 | 1,988 | 1,945 | 2,100 | 2,101 | −610 | −606 | 936 | 937 | 793 | 717 |
| radical.change | 2,037 | 2,044 | 1,958 | 1,913 | 2,075 | 2,066 | −601 | −601 | 928 | 929 | 790 | 714 |
| ami.change | 1,966 | 1,974 | 1,898 | 1,857 | 2,019 | 1,997 | −591 | −595 | 923 | 925 | 786 | 711 |
| Seconds to | | | | | | | | | | | | |
| time.to.max.s | 217.9 | 218.6 | 218.7 | 220.9 | 194.1 | 195.8 | 193.6 | 167.7 | 117.7 | 117.6 | 100.9 | 102.4 |
| t80.s | 12.9 | 12.1 | 12.0 | 12.6 | 8.9 | 8.6 | 3.6 | 3.1 | 3.3 | 3.0 | 2.7 | 2.5 |
| t90.s | 25.1 | 23.3 | 21.7 | 23.4 | 16.1 | 17.0 | 6.0 | 4.9 | 4.5 | 4.0 | 4.0 | 3.6 |
| radical.duration.s | 100.3 | 107.2 | 95.1 | 112.1 | 90.4 | 92.3 | 68.1 | 61.4 | 85.5 | 79.9 | 63.7 | 62.5 |
| ami.duration.s | 45.3 | 44.6 | 42.4 | 50.1 | 38.2 | 34.3 | 23.8 | 20.0 | 31.5 | 29.5 | 26.8 | 24.8 |

### Accuracy.

As shown in Table 2 below, the Coefficient of Variation (COV) for AMI as a percent of the maximum is less than 2%, suggesting a high degree of accuracy in a fraction of the time.

TABLE 2
Measure of precision by calibration method
Coefficient of Variation (St. Dev/Mean) - Measure of Precision

| | Carbon Monoxide | | | | | | Nitrogen Dioxide | | Oxygen | | | |
|---|---|---|---|---|---|---|---|---|---|---|---|---|
| Serial Number | 012A | 012B | 013A | 013B | 014A | 014B | 004A | 005A | 015A | 016A | 017A | 018A |
| Count of gas type | 132 | 110 | 141 | 110 | 53 | 52 | 95 | 107 | 61 | 63 | 117 | 125 |
| Output at zero gas | | | | | | | | | | | | |
| sensor.response.min | 5.3% | 4.2% | 6.2% | 5.8% | 2.6% | 2.6% | 1.9% | 0.2% | 0.3% | 0.3% | 4.0% | 14.1% |
| radical.min | 5.3% | 3.8% | 6.1% | 5.1% | 2.5% | 2.8% | 1.9% | 0.2% | 0.3% | 0.3% | 4.0% | 14.1% |
| ami.min | 5.3% | 3.8% | 6.0% | 5.0% | 2.6% | 2.6% | 1.9% | 0.2% | 0.3% | 0.3% | 4.0% | 14.1% |
| Sensor response at | | | | | | | | | | | | |
| sensor.response.max | 2.8% | 2.3% | 3.5% | 5.4% | 3.2% | 3.7% | 8.7% | 5.6% | 1.1% | 0.9% | 0.5% | 7.2% |
| sensor.response.at.t80 | 2.8% | 2.2% | 3.5% | 5.1% | 3.1% | 3.5% | 6.8% | 4.1% | 0.9% | 0.8% | 0.9% | 8.1% |
| sensor.response.at.t90 | 2.8% | 2.2% | 3.5% | 5.3% | 3.1% | 3.6% | 7.7% | 4.8% | 1.0% | 0.9% | 0.6% | 7.6% |
| radical.max | 2.7% | 2.3% | 3.5% | 5.0% | 3.2% | 3.8% | 8.6% | 5.5% | 1.3% | 1.1% | 0.6% | 7.2% |
| ami.max | 2.9% | 2.4% | 3.4% | 4.7% | 3.3% | 3.9% | 8.3% | 5.4% | 1.1% | 1.0% | 0.6% | 7.2% |
| radical.percent.of.max | 1.0% | 1.1% | 1.1% | 1.4% | 0.8% | 1.1% | 0.7% | 0.7% | 1.8% | 1.7% | 0.9% | 1.4% |
| ami.percent.of.max | 1.2% | 1.4% | 1.5% | 1.8% | 1.0% | 1.3% | 0.9% | 0.9% | 1.0% | 1.0% | 0.4% | 0.9% |
| Span | | | | | | | | | | | | |
| sensor.response.change | 3.6% | 3.3% | 4.3% | 7.7% | 4.4% | 4.9% | 16.5% | 12.0% | 3.0% | 2.7% | 8.6% | 15.5% |
| radical.change | 3.5% | 3.3% | 4.4% | 7.2% | 4.3% | 5.0% | 16.4% | 12.0% | 3.7% | 3.3% | 8.5% | 15.4% |
| ami.change | 3.6% | 3.4% | 4.3% | 6.8% | 4.5% | 5.2% | 16.2% | 11.9% | 3.3% | 2.9% | 8.5% | 15.6% |
| Seconds to | | | | | | | | | | | | |
| time.to.max.s | 25.3% | 22.1% | 24.5% | 23.9% | 21.7% | 19.8% | 23.9% | 39.1% | 34.2% | 35.5% | 33.9% | 39.9% |
| t80.s | 32.0% | 31.3% | 33.1% | 26.1% | 38.3% | 42.3% | 29.1% | 25.7% | 8.7% | 11.1% | 29.0% | 21.1% |
| t90.s | 27.1% | 30.3% | 31.0% | 26.0% | 42.1% | 46.6% | 33.8% | 35.0% | 19.2% | 12.8% | 25.0% | 19.9% |
| radical.duration.s | 35.5% | 34.3% | 35.5% | 34.7% | 30.7% | 40.7% | 50.0% | 56.8% | 50.0% | 33.7% | 37.0% | 35.7% |
| ami.duration.s | 23.2% | 22.8% | 19.1% | 26.5% | 21.4% | 19.9% | 27.6% | 30.8% | 17.9% | 17.8% | 17.8% | 20.3% |

In some embodiments, this alternative method to the RADiCal method discussed above, offers similar advantages to the RADiCal in terms of accuracy, self-referencing and speed, but is much less susceptible to changes in firmware and hardware configuration, and can be made computationally less intensive.

This method may also be used to identify another characterization point, such as an inflection point of the signal, as would be indicated by applying the AMI to the first difference of the signal. It is expected that there is a relationship between the maximum signal and the inflection point. The AMI provides a way of determining the inflection point.

As noted above, embodiments of the teachings herein may apply to the application of the calibration method to various types of sensors, such as, but not limited to, gas, temperature, humidity, vibration, pressure, motion, light, sound, particles, biosensors, etc. The target value each type of sensor may be a unit of measure typically used for the phenomena detected by that sensor. For example, the target value for a gas sensor may be a gas concentration level. For a temperature sensor, the target value is commonly a degrees centigrade level. For a humidity sensor, and the target value is commonly a relative humidity level. For a vibration sensor, the target value is commonly a rate of change in displacement per unit time level. For a pressure sensor, the target value is commonly a Pascal level. For a motion sensor or a light sensor, the target value is a frequency or luminosity. For a sound sensor, the target value is a change in sound pressure level (SPL). For particulate matter, the target value is commonly the number of micrograms per cubic meter (μg/m3) parts per million. For microbes, the target value is commonly measure in cell numbers or cell mass. It should be noted that other units of measurements may be used for each sensor, and a person skilled in the art would understand which unit of measurement to use in different circumstances.

A further embodiment of this disclosure is that temperature and humidity correction factors can be derived from the shape of the curve itself.

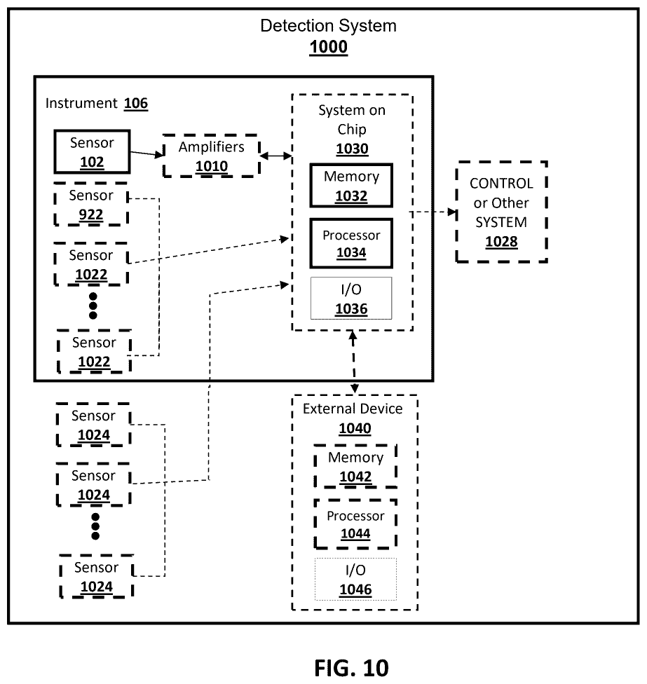

FIG. 10 illustrates, in a schematic diagram, another example of a detection system 1000, in accordance with some embodiments. As shown in FIG. 10, the detection system 1000 comprises an instrument 106 comprising a first sensor 102 (e.g., a gas sensor or other type of sensor), a memory 1032, a processor 1034 and an input/output (I/O) unit 1036. Logic corresponding to the calibration unit 104 may be stored in the memory 1032 as firmware and/or software. The memory 1032, processor 1034 and I/O unit 1036 may be included on a system on chip 1030.

The instrument 106 may optionally include additional sensors 1022 such as a gas sensor, a temperature sensor, a humidity sensor, a pressure sensor, a vibration sensor, or other types of sensors, all of which may be used to contribute to the estimation of gas concentrations and/or gain adjustments for calibration. The instrument 106 may also optionally include at least one amplifier 1010 to amplify signals corresponding to gas readings such that the small “random” variations may better detected.

The system 1000 may optionally include additional sensors 1024 such as a gas sensor, a temperature sensor, a humidity sensor, a pressure sensor, a vibration sensor, or other types of sensors, all of which may be used to contribute to the estimation of phenomena measurements (e.g., gas concentrations) and/or gain adjustments for calibration. The system 1000 may also optionally include a Control or other system 1028, and an external device 1040 comprising its own memory 1042, processor 1044 and I/O unit 1046. The external device 1040 may comprise a smart phone, tablet, computer or other computing device that may be in communication with the instrument. For example, the external device 1040 may comprise logic corresponding to the calibration unit 104 such that the external device 1040 control the calibration of the instrument 106.

FIG. 10 is a schematic diagram of a computing device 1100 such as a server. As depicted, the computing device includes at least one processor 1102, memory 1104, at least one I/O interface 1106, and at least one network interface 1108.

Processor 1102 may be an Intel or AMD x86 or x64, PowerPC, ARM processor, or the like. Memory 1104 may include a suitable combination of computer memory that is located either internally or externally such as, for example, random-access memory (RAM), read-only memory (ROM), compact disc read-only memory (CDROM). The memory 1104 may store instructions corresponding to methods 200 to 800. The processor 1102 may execute the instructions.

Each I/O interface 1106 enables computing device 1100 to interconnect with one or more input devices, such as a keyboard, mouse, camera, touch screen and a microphone, or with one or more output devices such as a display screen and a speaker.

Each network interface 1108 enables computing device 1100 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others.

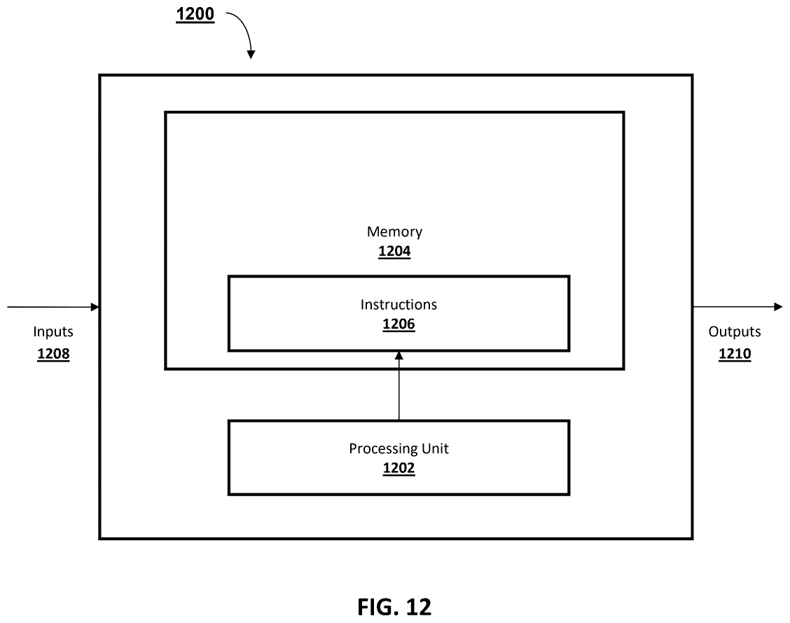

FIG. 12 shows a schematic representation of the calibration unit 104, as a combination of software and hardware components in a computing device 1200. The computing device 1200 may comprise one or more processing units 1202 and one or more computer-readable memories 1204 storing machine-readable instructions 1206 executable by the processing unit 1202 and configured to cause the processing unit 1202 to generate one or more outputs 1210 based on one or more inputs 1208. The inputs 1208 may comprise one or more signals representative of the inputs described in methods 200 to 800. The outputs 1210 may comprise one or more signals representative of the outputs described in methods 200 to 800.

Processing unit 1202 may comprise any suitable devices configured to cause a series of steps to be performed by computing device 1200 so as to implement a computer-implemented process such that instructions 1206, when executed by computing device 1200 or other programmable apparatus, may cause the functions/acts specified in methods 200 to 800 to be executed. Processing unit 1202 may comprise, for example, any type of general-purpose microprocessor or microcontroller, a digital signal processing (DSP) processor, an integrated circuit, a field programmable gate array (FPGA), a reconfigurable processor, other suitably programmed or programmable logic circuits, or any combination thereof.

Memory 1204 may comprise any suitable known or other machine-readable storage medium. Memory 1204 may comprise non-transitory computer readable storage medium such as, for example, but not limited to, an electronic, magnetic, optical, electromagnetic, infrared, or semiconductor system, apparatus, or device, or any suitable combination of the foregoing. Memory 1204 may include a suitable combination of any type of computer memory that is located either internally or externally to computing device 1200 such as, for example, random-access memory (RAM), read-only memory (ROM), compact disc read-only memory (CDROM) (or FLASH memory), electro-optical memory, magneto-optical memory, erasable programmable read-only memory (EPROM), and electrically-erasable programmable read-only memory (EEPROM), Ferroelectric RAM (FRAM) or the like. Memory 1204 may comprise any storage means (e.g. devices) suitable for retrievably storing machine-readable instructions 1206 executable by processing unit 1202.

The discussion provides example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

Although the embodiments have been described in detail, it should be understood that various changes, substitutions and alterations can be made herein.

Moreover, the scope of the present application is not intended to be limited to the particular embodiments of the process, machine, manufacture, composition of matter, means, methods and steps described in the specification.

As can be understood, the examples described above and illustrated are intended to be exemplary only.

What is claimed is:

1. A calibration system for calibrating an instrument, the calibration system comprising:
at least one sensor;
a processor; and
a memory comprising instructions which, when executed by the processor, configure the processor to:
obtain a series of sensor readings;
determine variations between changes in successive sensor readings from the series of sensor readings;
estimate a stabilization point of the sensor readings by identifying at least one sensor reading from the series of sensor readings at which the sum of increases between successive readings is approximately 50% of the sum of the absolute value of the differences between successive reading; and
adjust, based on the stabilization point, a parameter in the instrument that represents an association between sensor readings and a known physical quantity.

2. The calibration system as claimed in claim 1, wherein the stabilization point comprises a point where the change in the sensor reading reaches near zero.

3. The calibration system as claimed in claim 1, wherein the processor is configured to:
update a reading buffer with the series of sensor readings;
populating a derivative buffer based on the reading buffer data;
estimate a AMI of a last N sample derivative readings;
retrieve a AMI from a previous N sample of derivative.

4. The calibration system as claimed in claim 3, wherein the processor is configured to consider the AMI to be 0.5 if all of prior N derivative readings are less than the resolution value.

5. The calibration system as claimed in claim 3, wherein the processor is configured to determine that the observation is stable when its relevant statistic estimate is sufficiently close to a value representative of a stable signal.

6. The calibration system as claimed in claim 3, wherein the processor is configured to determine that the observation is extreme in an upward or downward direction (or at an inflection point) when its relevant statistic estimate is sufficiently close to a value representative of a rapidly changing signal.

7. The calibration system as claimed in claim 3, wherein the processor is configured to determine that the observation is unstable when it is neither stable nor extreme.

8. The calibration system as claimed in claim 1, wherein the processor is configured to:
associate a physical quantity with the sensor output as identified through the calibration process.

9. The calibration system as claimed in claim 1, wherein the processor is configured to:
associate a target value with the sensor output as identified through the calibration process.

10. A computer-implemented method of calibrating an instrument, the method comprising:
obtaining a series of sensor readings;
determining variations between changes in successive sensor readings from the series of sensor readings;
estimating a stabilization point of the sensor readings by identifying at least one sensor reading from the series of sensor readings at which the percentage of total recent movement which was in an upward direction falls within a certain threshold; and
adjusting, based on the stabilization point, a parameter in the instrument that represents an association between sensor readings and a known physical quantity.

11. The method as claimed in claim 10, wherein the stabilization point comprises a point where the change in the sensor reading reaches near zero.

12. The method as claimed in claim 10, comprising:
updating a reading buffer with the series of sensor readings;
populating a derivative buffer based on the reading buffer data;
estimating a relevant statistic based on the last N observations;
determining an interval where the relevant statistic of stable readings would fall;
determining one or more intervals where the relevant statistic of extreme readings would fall.

13. The method as claimed in claim 12, wherein the AMI is considered to be 0.5 if all of prior N derivative readings are less than the resolution value.

14. The method as claimed in claim 12, comprising determining that the observation is stable when its relevant statistic estimate is sufficiently close to a value representative of a stable signal.

15. The method as claimed in claim 12, comprising determining that the observation is extreme in an upward or downward direction (or at an inflection point) when its relevant statistic estimate is sufficiently close to a value representative of a rapidly changing signal.

16. The method as claimed in claim 12, comprising determining that the observation is unstable when it is neither stable nor extreme.

17. The method as claimed in claim 10, comprising:
associating a physical quantity value with the sensor output as identified through the calibration process.

18. The method as claimed in claim 10, comprising:
associating a target value with the sensor output as identified through the calibration process.

19. A calibration subsystem for calibrating an instrument, the calibration subsystem comprising:
a processor; and
a memory comprising instructions which, when executed by the processor, configure the processor to:
obtain a series of sensor readings from at least one sensor;
determine variations between changes in successive sensor readings from the series of sensor readings;
estimate a stabilization point of the sensor readings by identifying at least one sensor reading from the series of sensor readings at which the percentage of total recent movement which was in an upward direction falls within a certain threshold; and
adjust, based on the stabilization point, a parameter in the instrument that represents an association between sensor readings and a known physical quantity.
